(19) United States
(12) Patent Application Publication
(10) Pub. No.: US 2006/0115512 A1
(43) Pub. Date: Jun. 1, 2006

(54) Metallic structures incorporating bioactive materials and methods for creating the same

(75) Inventors: James C. Peacock III, San Carlos, CA (US); Richard L. Klein, Santa Rosa, CA (US); Michael J. Lee, Santa Rosa, CA (US)

Correspondence Address: PRESTON GATES & ELLIS LLP;ATTN: C. RACHAL WINGER, 925 FOURTH AVE, SUITE 9200, SEATTLE, WA, 98104-1158, US

(73) Assignee: Medlogics Device Corporation, Santa Rosa, CA (US)

(21) Appl. No.: 11/223,234

(22) Filed: Sep. 9, 2005

Related U.S. Application Data
(63) Continuation-in-part of application No. 10/724,453, filed on Nov. 28, 2003.

(51) Int. Cl. A61F 13/00

(52) U.S. Cl. 424/422

(57) ABSTRACT

Disclosed herein are methods to create medical devices and implantable medical devices with an electrochemically engineered porous surface that contains one or more bioactive materials to form bioactive composite structures. The bioactive composite structures are prepared using electrochemical codeposition methods to create metallic layers with pores that can be loaded with bioactive materials. In one use, the implantable medical devices of the present invention include stents with bioactive composite structure coatings.

### CROSS-REFERENCES TO RELATED APPLICATIONS

This application is a continuation-in-part of U.S. patent application Ser. No. 10/724,453, filed Nov. 28, 2003. All of these patent applications are herein incorporated by reference in their entirety for all purposes.

### FIELD OF THE INVENTION

The present invention relates generally to surfaces of implantable medical devices. More specifically, it pertains to an implantable medical device with an electrochemically engineered porous surface that contains within its pores one or more bioactive materials.

### BACKGROUND OF THE INVENTION

In many circumstances, it is beneficial for an implanted medical device to release a bioactive material into the body once the device has been implanted. Such released bioactive materials can enhance the treatment offered by the implantable medical device, facilitate recovery in the implanted area and lessen the local physiological trauma associated with the implant. Vascular stents are one type of device that has benefited from the inclusion of bioactive materials. Stents are ridged, or semi-ridged, tubular scaffoldings that are deployed within the lumen (inner tubular space) of a vessel or duct during angioplasty or related procedures intended to restore patency (openness) to vessel or duct lumens. Stents generally are left within the lumen of a vessel or duct after angioplasty or a related procedure to reduce the risk of restenosis, abrupt reclosure or re-occlusion. Including bioactive materials such as, for example and without limitation, rapamycin or paclitaxel on the surface of the implanted stent further helps to prevent restenosis, abrupt reclosure or re-occlusion (hereinafter “reclosure”).

One challenge in the field of implantable medical devices has been adhering bioactive materials to the surface of implantable devices such that the bioactive materials will be released once the device is implanted. One approach has been to include the bioactive materials in polymeric coatings. Polymeric coatings can hold bioactive materials onto the surface of implantable medical devices and release the bioactive materials via degradation of the polymer or diffusion into liquid (in this case the polymer is non-degradable). Degradable and non-degradable polymers such as polylactic acid, polyglycolic acid, and polymethylmethacrylate have been used in drug-eluting stents.

While polymeric coatings can be used to adhere bioactive materials to implanted medical devices, there are a number of problems associated with their use. First, it is difficult to predict the degradation kinetics of polymers. Consequently, it is difficult to predict how quickly a bioactive material in a polymeric coating will be released. If a drug releases from the polymeric coating too quickly or too slowly, the intended therapeutic effect may not be achieved. Second, in some cases, polymeric coatings produce pro-thrombotic and pro-inflammatory responses. These pro-thrombotic and pro-inflammatory effects lead to the necessity of prolonged antiplatelet therapies. Further, in the case of stents, these effects can exacerbate restenosis, the negative effect stents are designed to prevent. Third, adherence of a polymeric coating to a substantially different substrate, such as a stent's metallic substrate, is difficult due to differing characteristics of the materials (such as differing thermal expansion properties). The difficulty in adhering the two different material types often leads to inadequate bonding between the medical device and the overlying polymeric coating which can result in the separation of the materials over time. Such separation is an exceptionally undesirable property in an implanted medical device. Fourth, it is difficult to evenly coat a medical device with a polymeric coating. The uneven coating of a medical device can lead to unequal drug delivery across different portions of the device. This drawback is especially apparent in relation to small implantable medical devices, such as stents. Further, due to the viscosity of polymers during coating, it is difficult to evenly coat a medical device to faithfully replicate its form. Fifth, polymeric coatings are large and bulky relative to their bioactive material storage capacity. Sixth, when delivering a bioactive material to a patient over a longer time period, the bioactive material needs to be stabilized. Some polymeric coatings cannot provide a stable storage environment for the bioactive material, in particular when liquid, such as blood, is able to seep into the polymeric coating. Seventh, polymeric coatings, which by their nature have large pores, can protect microorganisms in the interstices of the polymeric coating, thus increasing the risk of infection. Finally, polymeric coatings remain on the medical device once the bioactive materials they contained have fully-eluted. Thus, the negative effects of the polymeric coating remain even when the bioactive materials are no longer providing continued treatment.

Sintered metallic structures can be used as an alternative to polymeric coatings. In a typical sintering process, small particles of metal are joined by an epoxy and then treated with heat and/or pressure to weld them together and to the substrate. A porous metallic structure has then been created. While effective in some instances, sintered metallic structures have relatively large pores. When a bioactive material is loaded into the pores of a sintered metallic structure, the larger pore size can cause the biologically active material to be released too quickly. As noted above, it would be desirable to have the ability to increase the bioactive material storage capacity in a bioactive composite material so that, for example, the bioactive material can be released to a patient over a long period of time.

While several alternative methods for coating stents and other implantable medical devices with bioactive materials have been proposed, these methods suffer from drawbacks including those resulting from processing limitations in relation to the underlying substrate or bioactive agent to be coated; inability to obtain even distribution of coatings or bioactive materials; problems with adhesion; biocompatibility issues (e.g. toxicity, or other adverse biological response); complexity of processing; size; density (and thus volume of drug that can be held and released); timing of drug release; high electrical impedance; low radiopacity; or an impact of the coating on the underlying substrate's intended function (e.g. mechanical properties, expansion characteristics, electrical surface conduction, radiopacity, etc.). Thus, notwithstanding certain benefits that may be provided by polymeric coatings, sintering or other alternative methods for coating implantable medical devices with bioactive materials, there is still room for improvement. Specifically, it would be beneficial if a coating process and matrix could be provided that overcomes one or more of the above-mentioned limitations.

### SUMMARY OF THE INVENTION

The present invention addresses many of the drawbacks associated with previously-available methods of loading bioactive materials onto implantable medical devices by providing a method to create small pores within a metallic layer created on the surface of an implantable medical device that can be loaded with bioactive materials. Loading bioactive materials into small pores created within a metallic layer on the surface of an implantable medical device is advantageous for many reasons. First, the deposited metals, unlike polymers, are not pro-thrombotic or pro-inflammatory. Because polymers are not used to carry the bioactive materials, once the bioactive materials have eluted from the implantable medical device, only bare metal, which is not pro-thrombotic or pro-inflammatory, is left behind. Thus, no negative effects of including the bioactive materials are left behind once the bioactive materials have fully eluted. Second, when a metallic layer is deposited onto an implantable medical device that is also made from a metal, the metallic layer and underlying device do not have substantially different characteristics, so the risk of separation is diminished significantly. Third, deposition of a metallic layer in accordance with the methods of the present invention allows for an even coating of implantable medical devices regardless of their size or geometry. Fourth, harsh processing conditions that may damage bioactive materials during the coating or loading process, are not required and the ability to control the percentage of bioactive materials present within the metallic layer can be easily controlled. Finally, the methods according to embodiments of the present invention are economical and scaleable, and are more cost-effective than other methods of forming bioactive composite structures.

Specifically, the methods of the present invention create pores within metallic layers by codepositing metal and erodable particles onto the surface of an implantable medical device electrochemically through electrolytic, electroless or electrophoretic codeposition processes. In another embodiment, bioactive materials are included within the formed metallic layer itself by codepositing metal, erodable particles and bioactive materials through electrochemical codeposition methods. After the electrochemical codeposition methods have been performed, the erodable particles can be selectively removed from the metallic layer thus leaving pores in the metallic layer (or metallic layer with bioactive materials) than can be post-loaded with bioactive materials. During the electrochemical codeposition methods of the present invention, the concentration of metals, erodable particles and/or bioactive materials can be varied over time to vary the amount of erodable particles (and resulting pores) or bioactive materials in different sublayers of the metallic layer. Thus, in this manner, different bioactive material elution profiles over time can be created.

In one embodiment of the methods of the present invention, the method comprises providing a bath comprising metal ions and erodable particles; contacting the bath and the substrate; forming a composite structure on the substrate using an electrochemical process; removing the erodable particles from the composite structure after the formation of the composite structure thus leaving pores in the structure; and loading at least one bioactive material into the pores thus forming a biocomposite structure.

In another embodiment of the methods of the present invention, the electrochemical process is an electrolytic codeposition process, an electroless codeposition process or an electrophoretic codeposition process.

In another embodiment of the methods of the present invention, the provided bath further comprises at least one bioactive material and the formed composite structure after the contacting is a bioactive composite structure.

In another embodiment of the methods of the present invention, the erodable particles are polytetrafluoroethylene polymer particles. In another embodiment of the methods of the present invention, the erodable particles are polytetrafluoroethylene oligomer particles. In another embodiment of the methods of the present invention, the erodable particles are tetrafluoroethylene-hexafluoropropylene copolymer particles. In another embodiment of the methods of the present invention, the erodable particles are tetrafluoroethylene-perfluoroalkyl vinyl ether copolymer particles. In another embodiment of the methods of the present invention, the erodable particles are fluorinated graphite particles. In another embodiment of the methods of the present invention, the erodable particles are fluorinated pitch particles. In another embodiment of the methods of the present invention, the erodable particles are graphite particles. In another embodiment of the methods of the present invention, the erodable particles are molybdenum disulfide particles. In another embodiment of the methods of the present invention, the erodable particles are boron nitride particles. In another embodiment of the methods of the present invention, the erodable particles are any combination of polytetrafluoroethylene polymer particles, polytetrafluoroethylene oligomer particles, tetrafluoroethylene-hexafluoropropylene copolymer particles, tetrafluoroethylene-perfluoroalkyl vinyl ether copolymer particles, fluorinated graphite particles, fluorinated pitch particles, graphite particles, molybdenum disulfide particles, and boron nitride particles.

In another embodiment of the methods of the present invention, the bath further comprises a low viscosity silicone glycol surfactant. In another embodiment of the methods of the present invention, the bath further comprises glycerol. In another embodiment of the methods of the present invention, the bath further comprises a low viscosity silicone glycol surfactant and glycerol.

In another embodiment of the methods of the present invention, the substrate is a stent.

In another embodiment of the methods of the present invention, a topcoat is formed over the biocomposite structure.

In another embodiment of the methods of the present invention, the method comprises providing a bath comprising metal ions and erodable particles in a first ratio; contacting the bath with a substrate; changing the ratio of metal ions and erodable particles in the bath after a specified period of time to form a second ratio (either in the same or a different bath); and forming a composite structure on the substrate using an electrochemical process, wherein the first ratio is such that the erodable particles are trapped within the structure formed by the metal ions depositing on the substrate in a first concentration and the second ratio is such that the erodable particles will be trapped within the structure formed by the metal ions depositing on the substrate in a second concentration thus forming a structure with different amounts of erodable particles found in different levels of the composite structure; removing the erodable particles from the composite structure thus forming pores in the composite structure; and loading at least one bioactive material into the pores thus forming a bioactive composite structure such that different amounts of bioactive materials are found at different levels of the biocomposite structure.

In another embodiment of the methods of the present invention, the provided bath further comprises at least one bioactive material in a first ratio and the formed composite structure after contacting is a bioactive composite structure and wherein after the contacting, the first ratio of the metal ions, erodable particles and at least one bioactive material is changed to form a second ratio thus altering the concentration of the erodable particles and the at least one bioactive material in the biocomposite structure.

The present invention also includes medical devices with biocomposite structures formed on their surface. In one embodiment of the medical device of the present invention, the medical device comprises a bioactive composite structure formed by providing a bath comprising metal ions and erodable particles; contacting the bath and the substrate; forming a composite structure on the substrate using an electrochemical process; removing the erodable particles from the composite structure after the formation of the composite structure thus leaving pores in the structure; and loading at least one bioactive material into the pores thus forming a biocomposite structure.

In another embodiment of the medical devices of the present invention, the electrochemical process is an electrolytic codeposition process, an electroless codeposition process or an electrophoretic codeposition process.

In another embodiment of the medical devices of the present invention, the bath further comprises at least one bioactive material and the formed composite structure after the contacting is a bioactive composite structure.

In another embodiment of the medical devices of the present invention, the erodable particles are polytetrafluoroethylene polymer particles. In another embodiment of the medical devices of the present invention, the erodable particles are polytetrafluoroethylene oligomer particles. In another embodiment of the medical devices of the present invention, the erodable particles are tetrafluoroethylene-hexafluoropropylene copolymer particles. In another embodiment of the medical devices of the present invention, the erodable particles are tetrafluoroethylene-perfluoroalkyl vinyl ether copolymer particles. In another embodiment of the medical devices of the present invention, the erodable particles are fluorinated graphite particles. In another embodiment of the medical devices of the present invention, the erodable particles are fluorinated pitch particles. In another embodiment of the medical devices of the present invention, the erodable particles are graphite particles. In another embodiment of the medical devices of the present invention, the erodable particles are molybdenum disulfide particles. In another embodiment of the medical devices of the present invention, the erodable particles are boron nitride particles. In another embodiment of the medical devices of the present invention, the erodable particles are any combination of polytetrafluoroethylene polymer particles, polytetrafluoroethylene oligomer particles, tetrafluoroethylene-hexafluoropropylene copolymer particles, tetrafluoroethylene-perfluoroalkyl vinyl ether copolymer particles, fluorinated graphite particles, fluorinated pitch particles, graphite particles, molybdenum disulfide particles, and boron nitride particles.

In another embodiment of the medical devices of the present invention, the bath further comprises a low viscosity silicone glycol surfactant. In another embodiment of the medical devices of the present invention, the bath further comprises glycerol. In another embodiment of the medical devices of the present invention, the bath further comprises low viscosity silicone glycol surfactant and glycerol.

In another embodiment of the medical devices of the present invention, the substrate is a stent.

In another embodiment of the medical devices of the present invention, a topcoat is formed over the biocomposite structures.

In another embodiment of the medical devices of the present invention, the medical device comprises a bioactive composite structure wherein the bioactive composite structure is formed by providing a bath comprising metal ions and erodable particles in a first ratio; contacting the bath with a substrate; changing the ratio of metal ions and erodable particles in the bath after a specified period of time to form a second ratio; and forming a composite structure on the substrate using an electrochemical process, wherein the first ratio is such that the erodable particles will be trapped within the structure formed by the metal ions depositing on the substrate in a first concentration and the second ratio is such that the erodable particles will be trapped within the structure formed by the metal ions depositing on substrate in a second concentration thus forming a structure with different amounts of erodable particles found in different levels of the composite structure; removing the erodable particles from the composite structure thus forming pores in the composite structure; and loading at least one bioactive material into the pores thus forming a bioactive composite structure such that different amounts of bioactive materials are found at different levels of the biocomposite structure.

In another embodiment of the medical devices of the present invention, the provided bath further comprises at least one bioactive material in a first ratio and the formed composite structure after the contacting is a bioactive composite structure and wherein after the contacting, the first ratio of metal ions, erodable particles and the at least one bioactive material is changed to form a second ratio thus altering the concentration of the erodable particles and bioactive materials in the biocomposite structure.

### DETAILED DESCRIPTION

I. Definitions

Some terms that are used herein are described as follows.

The term “bioactive material(s)” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials can include, without limitation, anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Exemplary drugs include, but are not limited to, antiproliferatives such as paclitaxel and rampamycin, everolimus, tacrolimus, de-saspartate angiotensin I, exochelins, nitric oxide, apocynin, gamma-tocopheryl, pleiotrophin, estradiol, heparin, aspirin and HMG-COA reductase inhibitors such as, but not limited to, atorvastatin, cerivastatin, fluvastatin, lovastatin, pravastatin, simvastatin, etc.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

The term “medical device” refers to an entity not produced in nature, which performs a function inside or on the surface of the human body. Medical devices include but are not limited to: biomaterials, drug delivery apparatuses, vascular conduits, stents, plates, screws, spinal cages, dental implants, dental fillings, braces, artificial joints, embolic devices, ventricular assist devices, artificial hearts, heart valves, venous filters, staples, clips, sutures, prosthetic meshes, pacemakers, pacemaker leads, defibrillators, neurostimulators, neurostimulator leads, and implantable or external sensors. Medical devices are not limited by size and include micromechanical systems and nanomechanical systems which perform a function in or on the surface of the human body. Embodiments of the invention include such medical devices.

The term “substrate” refers to any physical object that can be submerged in a bath and subjected to electrolytic, electroless or electrophoretic codeposition of metal ions and erodable particles.

The terms “implants” or “implantable” refers to a category of medical devices, which are implanted in a patient for some period of time. They can be diagnostic or therapeutic in nature, and long or short term.

The term “self-assembly” refers to a nanofabrication process of forming a material or coating, which proceeds spontaneously from a set of ingredients. A common self-assembly process includes the self-assembly of an organic monolayer on a substrate. One example of this process is the binding of linear organic molecules to a substrate. In this example, each molecule contains a thiol group (S—H moiety) and the thiol group of each molecule couples to the substrate while the other end of the molecule extends away from the substrate. The process of electroless deposition or codeposition, which continues spontaneously and auto-catalytically from a set of ingredients, can also be considered a self-assembly process.

The term “stents” refers to devices that are used to maintain patency of a body lumen or interstitial tract. There are two categories of stents; those which are balloon expandable (e.g., stainless steel) and those which are self expanding (e.g., nitinol). Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently stent diameters are limited to about 2 to 3 millimeters). For example, they will be used in the interstitium to create conduits between the ventricles of the heart and coronary arteries, or between coronary arteries and coronary veins. In the eye, stents are being developed for the Canal of Schlem to treat glaucoma.

The phrase “electrochemical process” as used herein means an electrolytic deposition process (also known as electroplating), an electroless deposition process, an electrophoretic deposition process or an electrolytic codeposition process, an electroless codeposition process, or an electrophoretic codeposition process. Deposition refers to deposition of a metal alone through an electrolytic, electroless or electrophoretic process (although, as will be understood by one of skill in the art, an electroless or electrophoretic process also involves ions of a reducing agent). A codeposition process refers to approximately concurrent deposition of metal and erodable particles or metal, erodable particles and bioactive materials through an electrolytic, electroless or electrophoretic process.

The term “solution” as used herein means any liquid in which an electrochemical process takes place and can be, without limitation, an electrolyte solution, an electrochemical solution and an electroless or electrophoretic bath.

The phrase “composite structure” as used herein refers to the material overlying a substrate that results from an electrochemical deposition process that does not include any bioactive materials.

The phrase “bioactive composite structure” as used herein refers to the material overlying a substrate that includes bioactive materials.

II. Methods of Manufacture

Embodiments of the invention include methods of coating substrates including implantable medical devices with bioactive materials to form bioactive composite structures. In one embodiment of the present invention, erodable particles are deposited with the metal. These particles are removed leaving pores which can then be loaded with bioactive materials.

A. Substrate and Substrate Preparation

The substrates of the present invention can be prepared in any suitable manner prior to forming a bioactive composite structure on its surface. For example, in one embodiment, a metallic layer with erodable particles is formed using an electrolytic, electroless or electrophoretic codeposition process (described more fully below). In these embodiments, the substrate surface can be sensitized and/or catalyzed prior to performing the electrochemical codeposition process (if the surface of the substrate is not itself autocatalytic). Metals such as tin (Sn) can be used as sensitizing agents. Many metals (e.g., nickel (Ni), cobalt (Co), copper (Cu), silver (Ag), gold (Au), palladium (Pd), platinum (Pt)) are good auto catalysts. Palladium, Pt, and Cu are examples of “universal” nucleation center forming catalysts. In addition, many non-metals are good catalysts as well.

Before creation of a metallic layer with erodable particles, the substrate also can be rinsed and/or precleaned if desired. Any suitable rinsing or pre-cleaning liquid or gas could be used to remove impurities from the surface of the substrate before creating the metallic layer with erodable particles. Also, in some embodiments involving electroless codeposition, distilled water can be used to rinse the substrate after sensitizing and/or catalyzing, but before performing the electroless process in order to remove loosely attached molecules of the sensitizer and/or catalyst.

Prior to performing an electrochemical codeposition process to create a metallic layer with erodable particles, the substrates of the present invention also can undergo an anodic process. In this process, the substrate is submerged in a hydrochloric acid bath. Current is passed through the hydrochloric acid bath, creating small pits in the substrate. Such pits promote adhesion. Also, a sensitizing agent and/or catalyst can be deposited on the substrate to assist in the creation of nucleation centers leading to the formation of the bioactive composite structure. Loosely adhered nucleation centers can also be removed from the surface of the substrate using, for example, a rinsing process.

A substrate also can be immersed in a “striking” bath as described in co-pending U.S. patent application Ser. No. 10/701,262 filed on Nov. 3, 2003, which incorporated by reference herein for all it contains regarding striking baths. Specifically, in a striking bath, a current is applied across the substrate causing metal ions to move to the device and plate the surface. This step causes an intermediate or “strike” layer to be formed on the surface of the substrate. Metal ions for this first striking bath are chosen to be compatible with the material making up the substrate itself. For example, if the underlying substrate is made of cobalt chrome, cobalt ions are used. It has been found that this strike layer improves overall adherence of the coating to the substrate as well as increasing the rate of codeposition during subsequent electrochemical codeposition processes. In one embodiment, when striking is performed, the substrate is rinsed with water prior to subsequent electrochemical codeposition processes.

Substrates of the present invention also can be immersed in a bath to form a seed layer (also disclosed in co-pending U.S. patent application Ser. No. 10/701,262 filed on Nov. 3, 2003, which is incorporated by reference herein for all it contains regarding seed layers). A seed layer is an electrolessly deposited metallic layer that is deposited before any codeposition processes. In one embodiment, a seed layer can be formed directly onto the surface of a substrate. In another embodiment, a seed layer can be formed on the surface of a strike layer. Metals for this seed layer also are chosen to be compatible with the material making up the substrate itself and/or the strike layer. A seed layer can be beneficial because it also can enhance the deposition and adhesion of a subsequently deposited composite or biocomposite structure. In one embodiment, when a seed layer is formed, the substrate is rinsed with water prior to subsequent electroless and/or electrophoretic deposition or codeposition.

B. Electrochemical Processes

After a substrate has been prepared according to any of the treatments described above, the substrate undergoes an electrochemical deposition or codeposition process to create a metallic layer with erodable particles. For purposes of the following discussion, deposition refers to deposition of metal alone (although, as will be understood by one of skill in the art, an electroless process also involves ions of a reducing agent) while codeposition refers to deposition of metal and erodable particles through an electrochemical process.

In electrolytic deposition, an anode and cathode are electrically coupled through an electrolyte. As current passes between the electrodes, metal is deposited on the cathode while it is either dissolved from the anode or originates from the electrolyte solution. Electrolytic deposition processes are well known in, for example, the metal plating industry and in the electronics industry.

An exemplary reaction sequence for the reduction of metal in an electrolytic deposition process is as follows:
M^{Z+}solubon+z^{e}→M_{tattice(electrode)}
In this equation, M is a metal atom, M^{Z+} is a metal ion with z charge units and e is an electron (carrying a unit charge). The reaction at the cathode is a reduction reaction and is the location where electrolytic deposition occurs. There is also an anode where oxidation takes place. To complete the circuit, an electrolyte solution is provided. The oxidation and reduction reactions occur in separate locations in the solution. In an electrolytic deposition process, the substrate is a conductor as it serves as the cathode in the process. Specific electrolytic deposition conditions such as the current density, metal ion concentration, and bioactive material concentration can be determined by those of ordinary skill in the art.

Electroless deposition processes can also be used in accordance with the methods of the present invention. In an electroless deposition process, current does not pass through a solution. Rather, the oxidation and reduction processes both occur at the same “electrode” (i.e., on the substrate). It is for this reason that electroless deposition results in the deposition of a metal and an anodic product (e.g., nickel and nickel-phosphorus).

In an electroless deposition process, the fundamental reaction is:
M^{Z+}solution+R_{ed solution}→M_{lattice (catalytic surface)}+Oxₛₒₗᵤₜᵢₒₙ

In this equation, R is a reducing agent, which passes electrons to the substrate and the metal ions. Ox is the oxidized byproduct of the reaction. In an electroless process, electron transfer occurs at substrate reaction sites (initially the nucleation sites on the substrate; these then form into sites that are tens of nanometers in size). The reaction is first catalyzed by the substrate and is subsequently auto-catalyzed by the reduced metal as a metal matrix forms.

The present invention also provides for electrophoretic codeposition methods. In electrophoretic codeposition methods, a slight charge is placed onto the substrate to be coated in order to attract positively-charged metal ions and/or positively-charged erodable particles. The amount of charge placed onto the substrate is not, however, sufficient to change the balance of the process into an electrolytic deposition (or electrolytic codeposition) only process as described above. Thus, the reactions occurring in the bath resemble electroless processes but with a migration of positively-charged materials toward the slightly-charged substrate.

Baths in which electroless or electrophoretic deposition take place can include at least metal ions and a reducing agent. The solvent that is used in these baths can include water so that the bath is aqueous. Generally, deposition conditions such as the pH, deposition time, bath constituents, and deposition temperature can be chosen by those of ordinary skill in the art.

Any suitable source of metal ions can be used in embodiments of the invention. The metal ions in the bath can be derived from soluble metal salts before they are in the bath. In solution, the ions forming the metal salts can dissociate from each other. Non-limiting examples of suitable metal salts for nickel ions include nickel sulfate, nickel chloride, and nickel sulfamate. Non-limiting examples of suitable metal salts for copper ions include cupric and cuprous salts such as cuprous chloride or sulfate. Non-limiting examples of suitable metal salts for tin cations can include stannous chloride or stannous floroborate. Other suitable salts useful for depositing other metals are known in the electroless deposition art. Different types of salts can be used if a metal alloy matrix is to be formed.

Reducing agents reduce the oxidation state of the metal ions in solution so that the metal ions deposit on the surface of the substrate as metal. Exemplary reducing compounds include, without limitation, boron compounds such as amine borane and phosphites such as sodium hypophosphite. The amount of the reducing agent used generally is not critical. In one embodiment, the reducing agent can be included in the range of about 0.05 to about 0.5 mole/liter. In another embodiment, the reducing agent can be included in the range of about 0.15 to about 0.3 mole/liter.

In the codeposition methods of the present invention, erodable particles that can be included, for example and without limitation, are fluoroplastics such as TFE (tetrafluoroethylene) polymers or oligomers, tetrafluoroethylene-hexafluoropropylene copolymers (FEP) and tetrafluoroethylene-perfluoroalkyl vinyl ether copolymers (PFA), fluorinated graphite ((CF)x), fluorinated pitch, graphite, molybdenum disulfide (MOS₂) and BN (boron nitride). These may be used singly or in combination. In one embodiment, the average erodable particle size is in the range of about 100 μm or below. In another embodiment, the average erodable particle size is in the range of about 0.1 μm to about 50 μm. In another embodiment, the average particle size is in the range of about 0.1 μm to 10 μm. In one embodiment, the amount of erodable particles to be added to the bath is, in total, in the range of about 100 g/L or below. In another embodiment, the amount of erodable particles to be added to the bath is about 0.1 g/L to about 100 g/L. In another embodiment, the amount of erodable particles to be added to the bath is about 0.1 g/liter to about 20 g/L.

Including low viscosity silicone glycol surfactants in the baths of the present invention can improve the aqueous dispersions of erodable particles which can improve the coating on the surface of the substrate. Thus, in one embodiment, the bath can include a low viscosity silicone glycol surfactant. In one embodiment the low viscosity silicone glycol surfactants can be polyoxyalkoxylated silicone glycol surfactants. In another embodiment, the polyoxyalkoxylated silicone glycol surfactants can have low viscosities of about 30 to about 60 centistokes when measured at 25° C. In another embodiment of the present invention the bath also can include glycerol which can produce improved aqueous dispersions of the erodable particles and works synergistically with the foregoing polyoxyalkoxylated silicone glycol surfactants to produce optimum results. In one embodiment, the silicone glycol surfactant can contain a polydimethylsiloxane backbone modified with the chemical attachment of polyoxyalkylene chains, such as that marketed by BASF, Inc. (Florham Park, N.J.) under the tradename Masil® SF-19. While the silicone glycol surfactant and the glycerol can each separately enhance dispersion, the combination of the silicone surfactant and glycerol in erodable particle dispersions can provide even more enhanced dispersions. The use of both materials can result in higher percentages of erodable particles in the deposit, generate less foam during mixing, and to result in lower particle size and range of particle size in the dispersion than when only one of the two additives is used.

Complexing agents also can be used in the baths of the present invention to hold the metal in solution. Complexing agents useful in accordance with the present invention include, for example and without limitation, carboxylic acids, oxycarboxylic acids and water-soluble salts thereof including, for example and without limitation, citric acid, malic acid, EDTA, malonic acid, phthalic acid, maleic acid, glutaric acid, lactic acid, succinic acid, adipic acid, acetic acid and the like, and water-soluble salts thereof. In one embodiment, chelating agents (e.g., citric acid, malic acid, EDTA, and water-soluble salts thereof) having intense metal complexing power, for example against nickel, can be used in a total amount of about 0.2 mole/L or below. In another embodiment, the same chelating agents can be used in a total amount of about 0.02 moles/L to about 0.2 moles/L. In another embodiment, the same chelating agents can be used in a total amount of about 0.05 to 0.1 mole/liter. In addition, malonic acid, lactic acid, succinic acid and water-soluble salts thereof are effective components when used to improve film appearance, pH buffering properties and throwing power. Accordingly, in one embodiment, these complexing agents can be used in combination with the intense chelating agents in an amount of about 2 moles/liter or below. In another embodiment, these complexing agents can be used in combination with the intense chelating agents in an amount of about 0.03 moles/L to about 1.5 moles/L. In another embodiment, these complexing agents can be used in combination with the intense chelating agents in an amount of about 0.05 moles/L to about 1 mole/L. In one embodiment, the total amount of the complexing agent is in the range of about 0.05 moles/L to about 2 moles/L. In another embodiment, the total amount of the complexing agent is in the range of about 0.1 moles/L to about 1.1 moles/L.

The bath also can include stabilizers and buffers. Buffers and stabilizers are used to increase bath life and improve the stability of the bath. Buffers are used to control the pH of the bath. Stabilizers can be used to keep the solution homogeneous. Exemplary stabilizers include lead, cadmium, copper ions, etc.

During the codeposition processes of the present invention, without being bound by theory, it is believed that nanometer-sized crystallites of metal first deposit onto the surface of the stent. Following this deposition of tens of nanometers of metal, metal ions and erodable particles codeposit onto the already deposited metal. Thus, the metal and erodable particles can deposit substantially simultaneously. When codepositing metal atoms and erodable particles, the erodable particles are incorporated into the metal matrix. These crystallites confine the erodable particles in the formed composite structure. By codepositing the erodable particles, the concentration of the erodable particles in composite structure can be high.

As an example of the methods of the present invention, an appropriate bath containing nickel sulfate and sodium hypophosphite can be created by using a Silverson® L4RT high shear mixer to create a dispersion containing about 12 gm of Fluorad FC 135; about 1 gm of Fluorad FC 170; about 12 gm of isopropyl alcohol; about 375 gm water and about 600 gm Zonyl® MP-1000 PTFE Powder. In this example, Fluorad FC 135 is a cationic fluorinated wetting agent and Fluorad FC 170 is a nonionic fluorinated wetting agent (both materials manufactured by the 3M Corporation, St. Paul, Minn.). The water, alcohol and wetting agents can be mixed together. With the mixer running at about 5000-6000 rpm, the PTFE powder can be slowly added in small amounts. Once all the PTFE powder has been wetted into the dispersion, mixing can be continued for about one hour. The temperature can then be allowed to rise to not more than about 60-65° C., and then cooled to room temperature.

In another example of the methods of the present invention, an Elnic 101C5 electroless nickel plating bath (comprising nickel sulfate, sodium hypophosphite, complexing agents for the nickel ions and ammonium hydroxide as a pH adjustor) can be prepared according to the Technical Data Sheet for this product making a 20% solution of the Elnic 101C5. The pH can be adjusted with ammonia to about 4.9-5.0. Then, about 2-12 mL/L of the PTFE dispersion can be added. The resulting plating bath can be used to plate electroless nickel/PTFE deposits.

Another useful bath composition in accordance with the present invention includes about 0.07 mole/L nickel sulfate (NiSO₄.7H₂O); about 0.22 mole/L sodium hypophosphite monohydrate; about 0.10 mole/L malic acid; about 0.30 mole/L malonic acid; about 0.85 mole/L adipic acid; a very small amount of stabilizer; a very small amount of thiourea; about 150 mg/L perfluoroalkyl quaternary ammonium iodide; about 150 mg/L ethylene oxide-added quaternary ammonium salt; and about 3.0 g/L PTFE (MP1100, available from E.I. du Pont de Nemours & Co., Wilmington, Del.) (average primary particle size=0.3 μm) at a pH of about 4.9 at 90° C.

As a further example of the methods of the present invention, a nickel-phosphorous alloy matrix can be electrolessly codeposited with erodable particles onto a substrate. The substrate can be activated and/or catalyzed (using, e.g., Sn and/or Pd) prior to metallizing. To produce this alloy matrix, a typical electroless deposition bath contains NiSO₄ (26 g/L), NaH₂PO₂ (26 g/L), Na-acetate (34 g/L) and malic acid (21 g/L). The bath can contain ions derived from the previously mentioned salts. Erodable particles also are in the bath. In one embodiment the erodable particles can be PTFE powder. In this example, sodium hypophosphite is the reducing agent and nickel ions are reduced by the sodium hypophosphite. The temperature of the bath is from about room temperature to about 95° C. depending on desired deposition time. The pH is generally from about 5 to about 7 (these processing conditions could be used in other embodiments). The substrate to be coated is then immersed in the bath and a composite structure including erodable particles can be formed on the substrate after a predetermined amount of time. The nickel ions in solution deposit onto the substrate as pure nickel (reduction reaction) along with nickel-phosphorous alloy (oxidation reaction); the erodable particles codeposit along the crystallite and grain boundaries of the deposited metal matrix to form a composite structure. Typically, the amount of phosphorous ranges from less than 1% to greater than 25% (mole %) and can be varied by techniques known to those skilled in the art.

The present invention also can use electrophoretic methods to codeposit metal and erodable particles onto the surface of a substrate such as an implantable medical device. In one embodiment of the electrophoretic codeposition methods of the present invention, the substrate can be sensitized in 37% hydrogen chloride (HCl) for approximately 3 to 10 minutes, and in one embodiment, for approximately 5 minutes. The substrate can then be activated with an electrolytic Ni-strike. The Ni-strike can occur in, for example and without limitation, a Woods strike bath (comprising approximately 240 g/L nickel chloride and approximately 320 ml/L HCl) or a Sulfamate strike bath (comprising approximately 320 g/L nickel sulfamate; approximately 30 g/L boric acid; approximately 12 g/L HCl; and approximately 20 g/L sulfamic acid). Appropriate submersion times in these strike baths can be approximately 1-4 minutes and in one embodiment 2.5 minutes. Activation also can include application of an approximately 50-200 mAmp current, and in one embodiment, a 100 mAmp current.

After activation in a strike bath, the substrate can have a small nickel-phosphorous (Ni—P) layer created on its surface by submerging the substrate in an electroless Ni—P bath comprising approximately 35.6 g/L nickel sulfamate; approximately 17 g/L sodium hypophosphate; approximately 15 g/L sodium succinate; approximately 1.3 g/L succinic acid for approximately 2 to 10 minutes (in one embodiment 5 minutes) at approximately 30-70° C. Following the creation of this Ni—P layer, the substrate can be mounted on a masking electrode and immersed in, in a non-limiting example, an electrophoretic Ni—P-surfactant-erodable-particle solution. Submersion in this bath can occur for approximately 20 to 60 minutes (in one embodiment for 30 minutes) at approximately 30-50° C. (in one embodiment 50° C.) with a current of approximately 0.1-20 mAmp (in one embodiment 5 mAmp).

In embodiments of the present invention employing electrophoretic processes, materials can be given a positive charge by coupling a cationic or zwitterionic surfactant to the material. Non-limiting examples of cationic surfactants that can be used in accordance with the present invention include hexadecyl trimethyl ammonium bromide (HTAB), benzethonium chloride (BZTC) and cationic cyclodextrin complexes such as, without limitation, N,N-diethylaminoethyl-β-cyclodextrin and 2,3-Di-(N,N-diethylaminoethyl)-N-amino-2,3-deoxy-β-cyclodextrin. A suitable example of a zwitterionic surfactant that can be used in accordance with the present invention includes, without limitation 3-[(3-cholamido-propyl)-dimethyl-ammonio]-1-propanesulfonate (CHAPS).

Dispersing agents also can be used in accordance with the present invention. Anionic dispersing agents that can be used in accordance with the present invention include sodium lignosulfonate, sodium naphthalene sulfonate-formaldehyde condensate (“Lomar D”), sodium polystyrene sulfonate (“Flexan 130”), polyacrylic acid (Acumer 9400 and Good-Rite K-732) and organic phosphate ester (Emphos CS-1361). Nonionic dispersing agents that can be used in accordance with the present invention include, without limitation, aliphatic alcohol ethoxylate (Atlas G5000), ethylene oxide-propylene oxide block copolymer (HLB=17.0; Pluronic P65) and polyoxyethylene (20) monolaurate (HLB=16.7; Tween 20™). Cationic dispersing agents that can be used in accordance with the present invention include, without limitation, dimethyl dicoco ammonium chloride (Arquad® 2C-75, Akzona Inc., Enka, N.C.) and N-alkyl(soya)trimethyl ammonium chloride (Arquad® S-50, Akzona Inc., Enka, N.C.). A zwitterionic dispersing agent that can be used in accordance with the present invention includes, without limitation, palmitamidopropylbetaine (Scheercotaine PAB).

Wetting agents also can be used in accordance with the present invention. Anionic wetting agents that can be used in accordance with the present invention include, without limitation, sodium lauryl sulfate, sodium dioctyl sulfosuccinate (“aerosol otb”), sodiumdodecyl benzene sulfonate (“witconate 90”) and sodium isopropyl naphthalene sulfonate (“aerosol OS”). Nonionic wetting agents that can be used in accordance with the present invention include, without limitation, secondary alcohol ethoxylate (“tergitol® 15-5-5”; Union Carbide Chemicals & Plastics Technology Corp., Danbury, Conn.) and pluronic L 62 (a block copolymer of propylene oxide and ethylene oxide).

The previously-described methods of the present invention describe codepositing metal and erodable particles. In another embodiment of the present invention, at least one bioactive material can also be included in the bath. When bioactive materials are also included in a bath, these materials will also codeposit with the metal and erodable particles. In these embodiment of the present invention, the ultimately formed biocomposite structure contains bioactive materials in the pores created by removal of the erodable particles (described more fully below) and within the metal structure surrounding the pores.

In another embodiment of the methods of the present invention, the amount of erodable particles and/or bioactive materials included in a bath can be varied throughout the codeposition processes. Varying the concentration of erodable particles and/or bioactive materials during the coating process allows the creation of differing resulting pore and bioactive material concentration layers on the substrate thus allowing different elution profiles over time. In addition, different porosities also can be included in different deposited layers on the substrate also allowing different elution profiles over time.

The metallic matrix of the bioactive composite structure formed during the codeposition methods of the present invention can include any suitable metal. The metal in the metallic matrix can be the same as or different from the substrate metal (if the substrate is metallic). The metallic matrix can include, for example, noble metals or transition metals. Suitable metals include, but are not limited to, nickel, copper, cobalt, palladium, platinum, chromium, iron, gold, and silver and alloys thereof. Examples of suitable nickel-based alloys include nickel-chromium, nickel-phosphorous, and nickel-boron. Any of these or other metallic materials can be deposited using a codeposition process. Appropriate metal salts can be selected to provide appropriate metal ions in the bath for the metal matrix that is to be formed.

After contacting the solution or bath, a composite structure or a bioactive composite structure is formed on the substrate using a codeposition process. Whether the structure is a composite structure or a bioactive composite structure at this stage depends on whether a bioactive material is included in the solution or bath during the initial immersion. After forming the composite or biocomposite structure, the structure/substrate combination is removed from the solution or bath and subjected to subsequent processing as desired.

C. Subsequent Processing

After electrochemical codeposition onto the surface of a substrate, the device can be processed further to alter its clinical features.

All embodiments of the present invention include erodable particles deposited with metal after the described electrochemical processes (while some also include the codeposition of bioactive materials along with the erodable particles and metal). In the embodiments of the present invention, the erodable particles are removed after the electrochemical processes. In one embodiment, the erodable particles can be removed by calcinations in which the temperature is ramped at approximately 0.2° C./minute to approximately 300° C., where it is maintained for approximately 30 minutes before cooling. The erodable particles of the present invention also can be removed by chemical oxidation or solvent dissolution at room temperature. In another embodiment, the erodable particles can be removed with ultraviolet light. In another embodiment of the present invention, the erodable particles can be removed with Tetra-Etch® (W. L. Gore & Associates, Inc., Newark, Del.). When the erodable particles are removed, pores are left behind which can then be filled with another substance (e.g. a bioactive material).

Methods to load bioactive materials into pores are known in the art. One such method is described in detail. In this method, a bioactive material is added to a first fluid. The bioactive material is dispersed throughout the first fluid so that it is in a true solution, saturated or supersaturated with the solvent or suspended in fine particles in the first fluid. If the bioactive material is suspended in particles in the first fluid, the pore size and the diameter of the opening of the pores are to be sufficiently large in comparison to the size of the particles to facilitate loading and unloading of the pores of the substrate.

The first fluid can be virtually any solvent that is compatible with the bioactive material. A suitable first fluid typically has a high capillary permeation. Capillary permeation or wetting is the movement of fluid on a solid substrate driven by interfacial energetics. Capillary permeation is quantitated by a contact angle, defined as the angle at the tangent of the first fluid droplet in fluid phase that has taken an equilibrium shape on a solid surface. A low contact angle means a higher wetting liquid. A suitably high capillary permeation corresponds to a contact angle less than about 900.

A high capillary permeation and a viscosity not greater than about ten centipoise allows the first fluid to penetrate into the pores of the substrate more quickly, eliminating a requirement to apply the first fluid to the substrate for a prolonged period of time. The first fluid can be volatile, facilitating its evaporation. Useful examples of some first fluids include, but are not limited to, acetone, ethanol, methanol, isopropanol, tetrahydrofuran, and ethyl acetate. The first fluid is applied to a porous substrate, for example by immersing or spraying the solvent in procedures that are well-known to one having ordinary skill in the art.

The first fluid is applied for a predetermined period of time, the specific time depending on the capillary permeation and viscosity of the first fluid, the volume of the pores, and the amount of bioactive materials to be deposited. Therapeutic parameters such as the concentration of the bioactive material in the solvent and dosages depend on the duration of local release, the cumulative amount of release, and desired rate of release. Correlations and interrelations between the therapeutic parameters are well-known to one having ordinary skill in the art and are simply calculated.

After applying the first fluid for a selected duration, the first fluid is removed from the substrate. In one example, the first fluid is removed by evaporation in ambient pressure, room temperature, and anhydrous atmosphere and/or by exposure to mild heat (e.g., 60° C.) under a vacuum condition.

After removal from the first fluid, the substrate typically has a clustered or gross formation of bioactive material gathered on its surface. The cluster is generally removed by immersing the substrate in a second fluid and agitating the substrate via mechanical perturbation techniques, such as vortexing or vigorous shaking. The second fluid is a non-solvent so that the bioactive material does not significantly dissolve in the second fluid. The non-solvent second fluid can have a low capillary permeation or a contact angle greater than about 90° and a viscosity not less than about 0.5 centipoise so that the second fluid is not capable of significantly penetrating into the pores during the process of agitation. Examples of a second fluid include, but are not limited to, saturated hydrocarbons or alkanes, such as hexane, heptane, and octane.

After immersion in the second fluid, the substrate is rinsed in a third fluid. The third fluid is typically a solvent to facilitate dissolution of the bioactive material. The third fluid generally has a low capillary permeation, corresponding to a contact angle greater than about 90°. The third fluid has a viscosity of not less than about 1.0 centipoise and is therefore incapable of significantly penetrating into the pores during the rinsing stage. In one embodiment, the third fluid can be highly volatile, for example having a boiling point of not greater than about 60° C. at 1 atm. Accordingly, the third fluid is capable of rapidly evaporating. Rapid evaporation of the third fluid causes the third fluid to be removed from the substrate prior to any significant penetration of the third fluid in the pores. A useful example of a highly volatile third fluid includes, but is not limited to, Freon® (Freon® is a registered Trademark of E.I. du Pont de Nemours and Co., Wilmington, Del.).

Rinsing with the third fluid is conducted rapidly for example in a range from 1 second to about 15 seconds, the exact duration depending on the solubility of the bioactive material in the solvent. Extended duration of exposure of the third fluid to the substrate may lead to the penetration of the third fluid into the pores.

The rinsing step is repeated, if desired, until all traces of bioactive material are removed from the surface of the substrate. Useful examples of third fluids include, but are not limited to, dimethylsulfoxide (DMSO), water, DMSO in an aqueous solution, glyme, and glycerol. The third fluid is removed from the substrate body using a technique such as evaporation in ambient pressure, room temperature and anhydrous atmosphere and/or by exposure to mild heat (e.g., 60° C.) under vacuum condition. The first, second and third fluids are selected to not affect the characteristics and composition of the bioactive material adversely.

In some embodiments, a surface of the substrate is coated with at least one bioactive material in addition to having at least one bioactive material deposited in the pores. A coating of bioactive material on the surface of the substrate is formed by adding the bioactive material to the third fluid rinse. The bioactive material is dispersed through the third fluid to form a true solution with the third fluid, rather than a dispersion of fine particles.

Alternative methods that can be used to load bioactive materials into the pores of the present invention include high pressure loading. In this method, the substrate is placed in a bath of the desired drug or drugs and subjected to high pressure or, alternatively, subjected to a vacuum. In the case of the vacuum, the air in the pores of the metal stent is evacuated and replaced by the drug-containing solution. Additional methods of loading bioactive materials into pores are disclosed in U.S. Pat. No. 6,379,381 issued to Hossainy et al., which is hereby incorporated by reference in its entirety.

If desired, a topcoat can be formed on the bioactive composite structures of the present invention. The topcoat can include any suitable material and can be in any suitable form. It can be amorphous or crystalline, and can include a metal, ceramic, etc. The topcoat can also be porous or solid (continuous).

The topcoat can be deposited using any suitable process. For example, the above-described processes (e.g., electrochemical deposition or codeposition) could be used to form the topcoat or another process can be used to form the topcoat. Alternatively, the topcoat could be formed by processes such as, but not limited to, dip coating, spray coating, vapor deposition, etc.

In some embodiments, the topcoat can improve the properties of the bioactive composite structure. For example, the topcoat can include a membrane (e.g., collagen type 4) that is covalently bound to the bioactive composite structure. The topcoat's function can be to induce endothelial attachment to the surface of a bioactive composite structure, while the bioactive material in the bioactive composite structure diffuses from below the topcoat. In another embodiment, a growth factor such as endothelial growth factor (EGF) or vascular endothelial growth factor (VEGF) is present in a topcoat that is on a bioactive composite structure. The growth factor is released from the topcoat to induce endothelial growth while the bioactive composite structure releases an inhibitor of smooth muscle cell growth.

In yet another embodiment of the present invention, the topcoat can improve the radiopacity of a medical device which includes the bioactive composite structure, while the underlying bioactive composite structure releases molecules to perform another function. For example, drugs can be released from the bioactive composite structure to prevent smooth muscle cell overgrowth, while a topcoat on the bioactive composite structure improves the radiopacity of the formed medical device. Illustratively, a topcoat comprising nickel-chromium and/or gold can be deposited on top of a bioactive composite structure comprising nickel-phosphorous to enhance the radiopacity of a device incorporating the bioactive composite structure. Underneath the topcoat, a smooth muscle cell inhibitor such as sirolimus can be released over a 30-60 day time period from the bioactive composite structure.

The topcoat can also be used to alter the release kinetics of the bioactive material in the underlying bioactive composite structure. For example, an electroless nickel-chrome, nickel-phosphorous, or cobalt-chrome coating without bioactive material can serve as a topcoat. This would require the bioactive material to travel through an additional layer of material before entering the surrounding environment, thereby delaying the release of the bioactive material. The release kinetics of the formed medical device can be adjusted in this manner.

In yet another embodiment of the present invention, the topcoat that is on the bioactive composite structure can be a self-assembled monolayer. The thickness of the self-assembled monolayer can be less than 1 nanometer (i.e., a molecular monolayer) in some embodiments. In one example, a thiol based monolayer can be adsorbed on a nickel matrix of a bioactive composite structure through the thiol functional group and can self-assemble on the nickel matrix. The introduction of the self-assembled monolayer can permit different surface ligands to be used with the bioactive composite structure. That is, various ligands or moieties can be attached to the ends of the molecules in the monolayer that extend away from the bioactive composite structure.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention can be combined with any one or more other features of any other embodiment of the invention, without departing from the scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method comprising:
providing a bath comprising metal ions and erodable particles;
contacting said bath with a substrate;
forming a composite structure on said substrate using an electrochemical process;
removing said erodable particles from said composite structure after said formation of said composite structure thus leaving pores in said structure; and
loading at least one bioactive material into said pores thus forming a biocomposite structure.

2. The method according to claim 1, wherein said electrochemical process is selected from the group consisting of electrolytic processes, electroless processes and electrophoretic processes.

3. The method according to claim 1, wherein said bath further comprises at least one bioactive material and said formed composite structure after said contacting is a bioactive composite structure.

4. The method according to claim 1, wherein said erodable particles are selected from the group consisting of polytetrafluoroethylene polymer particles, polytetrafluoroethylene oligomer particles, tetrafluoroethylene-hexafluoropropylene copolymer particles, tetrafluoroethylene-perfluoroalkyl vinyl ether copolymer particles, fluorinated graphite particles, fluorinated pitch particles, graphite particles, molybdenum disulfide particles, boron nitride particles and combinations thereof.

5. The method according to claim 1, wherein said bath further comprises a low viscosity silicone glycol surfactant.

6. The method according to claim 1, wherein said bath further comprises glycerol.

7. The method according to claim 1, wherein said bath further comprises a low viscosity silicone glycol surfactant and glycerol.

8. The method of claim 1, wherein said substrate is a stent.

9. The method of claim 1, further comprising forming a topcoat over said biocomposite structure.

10. A medical device comprising a bioactive composite structure wherein said bioactive composite structure is formed by:
providing a bath comprising metal ions and erodable particles;
contacting said bath with a substrate;
forming a composite structure on said substrate using an electroless process;
removing said erodable particles from said composite structure after said formation of said composite structure thus leaving pores in said structure; and
loading at least one bioactive material into said pores thus forming a biocomposite structure.

11. The medical device according to claim 10, wherein said bath further comprises at least one bioactive material and said formed composite structure after said contacting is a bioactive composite structure.

12. The medical device according to claim 10, wherein said erodable particles are selected from the group consisting of polytetrafluoroethylene polymer particles, polytetrafluoroethylene oligomer particles, tetrafluoroethylene-hexafluoropropylene copolymer particles, tetrafluoroethylene-perfluoroalkyl vinyl ether copolymer particles, fluorinated graphite particles, fluorinated pitch particles, graphite particles, molybdenum disulfide particles, boron nitride particles and combinations thereof.

13. The medical device according to claim 10, wherein said bath further comprises a low viscosity silicone glycol surfactant.

14. The medical device according to claim 10, wherein said bath further comprises glycerol.

15. The medical device according to claim 10, wherein said bath further comprises a low viscosity silicone glycol surfactant and glycerol.

16. The medical device according to claim 10, wherein said substrate is a stent.

17. The medical device according to claim 10, further comprising forming a topcoat over said biocomposite structure.

18. A method comprising:
providing a first bath comprising metal ions and erodable particles wherein said metal ions and said erodable particles in said first bath are provided at a first ratio;
contacting a substrate with said first bath;
forming a composite structure with a first concentration of metal ions and erodable particles on said substrate using an electrochemical process;
altering said first ratio between said metal ions and said erodable particles to form a second ratio;
continuing to form said composite structure on said substrate using an electrochemical process but with a second concentration of metal ions and said erodable particles;
removing said erodable particles from said composite structure after said formation of said composite structure thus leaving pores in said structure; and
loading at least one bioactive material into said pores thus forming a biocomposite structure.

19. The method according to claim 18, wherein said electrochemical process is selected from the group consisting of electrolytic processes, electroless processes and electrophoretic processes.

20. The method according to claim 18, wherein said first and/or said second bath further comprises at least one bioactive material and wherein said composite structure is a biocomposite structure.
